(19) United States
(12) Patent Application Publication
(10) Pub. No.: US 2007/0129610 A1
(43) Pub. Date: Jun. 7, 2007

(54) Method of providing automated medical assistance

(75) Inventor: John R. Squilla, Rochester, NY (US)

Correspondence Address: Mark G. Bocchetti;Patent Legal Staff, Eastman Kodak Company, 343 State Street, Rochester, NY, 14650-2201, US

(73) Assignee: Eastman Kodak Company

(21) Appl. No.: 11/295,219

(22) Filed: Dec. 6, 2005

(51) Int. Cl. A61B 5/00

(52) U.S. Cl. 600/300

(57) ABSTRACT

A method of automatically providing users with appropriate levels of assistance at a walk-up medical station or kiosk (200) using hierarchical levels of support (130, 135, 140) based on intelligent contextual awareness of events, combined with stored historical user information (230).

### FIELD OF THE INVENTION

The present invention relates to providing automated assistance at a walk-up medical kiosk using hierarchical levels of support based on intelligent contextual awareness of events as they are occurring.

### BACKGROUND OF THE INVENTION

It is desirable to provide users of a walk-up medical station with a multitude of support options and levels of support as they interact with the station. An example of the current state of the art is automated teller machines (ATM), which have an integrated telephone and a video camera for support and security. Customized assistance, however, is not provided at these machines. Other computer assistance systems provide technical support when a user encounters software bugs, program errors, or disk problems. These systems typically provide limited interactive support based on previously documented problems along with the ability to report problems via e-mail or a product support website. These systems do not provide interactive real-time assistance that is tailored to the specific problem that the user has encountered.

Telemedicine systems (medical applications of computer-based teleconferencing), by their nature, are 2-way communications with built-in levels of support between the customer, on one side of the electronic conversation, and the medical provider, on the other side. In medical applications, especially those where unassisted or minimally assisted medical tests are performed, there can be many situations that require some additional information or remedial actions to be taken. Traditional methods of providing additional support include help screens or animated avatars to provide instructions to the user. It would be more appealing to provide users with support that utilizes awareness of the events taking place at the station, stored customer information, and analysis of medical test results to automatically engage the appropriate support solution personalized for that user and appropriate for the particular session or event.

The present invention allows for the automated identification of the presence of a customer at a medical station. RFID tags are well known in the art as a means of detecting a presence as the tag approaches a detection mechanism searching for the tag.

### SUMMARY OF THE INVENTION

Briefly, according to one aspect of the present invention, automated assistance at a remote medical testing system uses hierarchical levels of support based on intelligent use of history data and contextual awareness of events as they are occurring.

A remote medical testing system may include a medical kiosk, a remote medical station, home medical monitoring, or a home testing system. These unassisted or minimally assisted systems supply key information on testing and/or conditions important to a patient, while providing a more convenient, private or faster means of gathering the information. Traditional user support for these systems includes answers to frequently asked questions and canned responses to typically encountered problems. The current invention predetermines the nature of needed assistance beyond this traditional support and automatically links the necessary support customized to the patient, in the form of a person, electronic information or other types of assistance such as brochures or e-mails.

### BRIEF DESCRIPTION OF THE DRAWINGS

FIG. 1 is a schematic of types of assistance available for a medical kiosk according to the present invention.

FIG. 2 is a schematic systems diagram according to the present invention.

FIG. 3 is a flowchart of a hierarchical assistance system for a medical kiosk according to the present invention.

FIG. 4 is a chart showing automated assistance support levels.

FIG. 5 is a schematic of a system according to the present invention.

### DETAILED DESCRIPTION OF THE INVENTION

The present invention will be directed in particular to elements forming part of, or in cooperation more directly with, a method and apparatus in accordance with the present invention. It is to be understood that elements not specifically shown or described may take various forms well known to those skilled in the art.

Electronic medical records (EMR) are well known in the industry. An electronic patient record (EPR) is similar and for this invention is considered to be the same.

Unassisted medical kiosks exist in the marketplace and provide basic vital statistics monitoring such as patient heart rate and blood pressure. See LifeClinic at www.LifeClinic.com. U.S. Patent Application Publication No. 2004/0044560 (Giglio et al.) discusses a device to test and output the personal data (fat analysis) of a user to a computer processor. U.S. Pat. No. 6,692,436 (Bluth et al.) teaches a health kiosk that provides blood pressure testing, a health and fitness evaluation, and a medication encyclopedia. Other unassisted kiosks aid a user in diagnosing a condition by using question and answer scripts to reach a diagnostic conclusion. U.S. Pat. No. 6,641,532 (Iliff) teaches the art of conducting an automated diagnostic session with a patient, using a plurality of disease scripts, a patient medical record, and a disease engine to process the script and route the changes to the medical record. Staffed medical kiosks also exist that provide a nurse to check on certain ailments. See MinuteClinic at www.MinuteClinic.com.

The above remote medical test systems provide convenient medical services to consumers with improved accessibility over visits to a doctor's office. However, the unassisted systems are limited in their ability to provide comprehensive diagnostic services due to the lack of secure access to patient medical records, including doctor's orders, prescription information and individual patient history, and the inability to perform diagnostic tests beyond basic vital statistic analysis or question and answer scripts. Although assisted systems can provide more diagnostic tests for patients, they are limited in convenience by their hours of operation, limited number of locations, and limited access to electronic patient records.

A need exists in the marketplace to further extend the utility of remote medical testing systems to provide a greater variety of support for their users and to do so with a minimum amount of involvement from a support staff. The number of outcomes and expectations from these systems allow for many steps to be automated. Since they can be located in different environments, for example nursing homes, pharmacies, malls, doctors' offices, hospitals, and military bases, the level of on-site support can vary. It is well known in the art that teleconferencing, by its very nature, allows for a provider to support the customer directly, even when the provider is located remotely from the system's user. It is also known that on-line computer support allows remote support by allowing a support person to remotely take over a networked workstation and remedy a problem. With medical tests, this can be taken further by allowing software and intelligent agents to analyze the different parameters of a user, their interface with the system, results of their tests, and the combination of these elements.

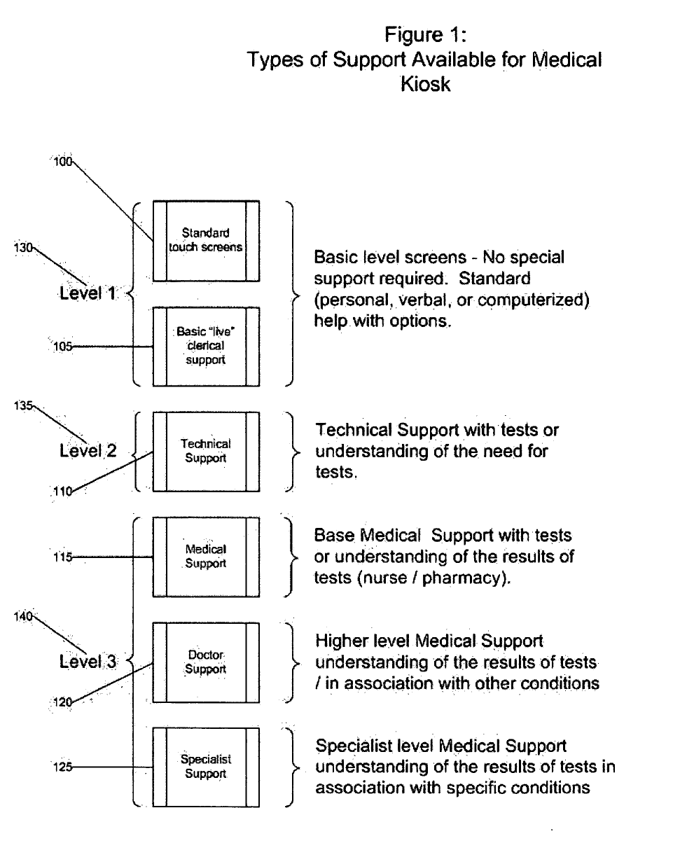

FIG. 1 shows one embodiment of how levels of support can vary. Level 1 Support 130 illustrates traditional support. Level 1 Support includes assistance for system failures 100 and for a lack of a basic understanding of the function of the system. This lack of understanding can be remedied by on-site clerical assistance 105 or via a traditional teleconference. One such example is the airline kiosk that has started to replace the traditional ticket desks. On-site clerical assistance from the airline is there to help people who have not used the kiosks before.

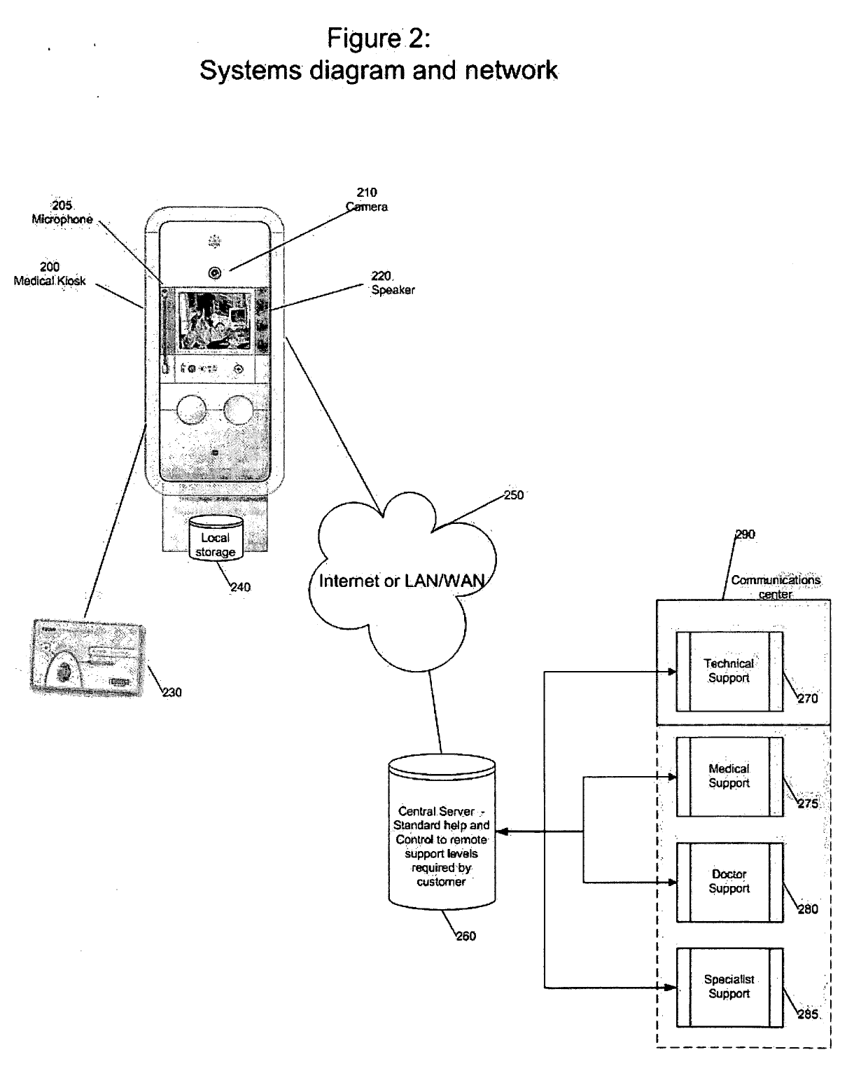

The current invention starts at Level 2 Support 135. Level 2 Support 135 involves technical assistance with the medical tests. In commonly-assigned co-pending U.S. patent application Ser. No. 10/995,676 the types of tests that can be performed at a remote medical testing system are explained. These tests have some consumable components associated with them as well as an interface to the testing system itself. If a customer is having problems with the test, a button (or screen click) on the system is available for support. Alternatively, a time delay can indicate to the system that there is a problem if a pre-determined time frame has elapsed and the customer has not provided the necessary inputs. Or, the customer may want more information about a test, for example, its side effects or medications that could affect the test results. A patient record 230, shown in FIG. 2, is important to automated support.

The patient record 230 can be embodied by several means. Detailed discussion of patient record embodiments can be found in commonly assigned co-pending U.S. patent application Ser. No. 10/991,553. A patient record 230 may be stored on a variety of media and include types of previously performed tests, results of these past tests, and user support required to perform these prior medical tests at the current medical testing system or another system.

With patient record information, it is possible to determine which type of support the patient requires. The Level 2 Support can include a simple reminder of what to do, step-by-step instructions on the screen, or a personal interview. This personal interview can be done by on-site staff or via an interaction with a trained support person using the conferencing capability of the system. Support can be provided at any level via computer screens with choices being made via mouse or touch screen, or via a personal interface via the teleconference system whereby the choices are made verbally by the customer to the support person, or by any combination of these. Personal preferences may be on the electronic patient record 230 and updated if the preferences change after an individual session. The medical testing system may also query the user as to which type of support they prefer for an individual session.

Level 3 Support 140 requires medical assistance to be delivered to the customer. This medical assistance is not physical in nature, but includes basic descriptions of the medical tests and results 115; support for next steps to be taken based on the test results 120, and further coordination with a medical specialist 125, when needed.

A basic description of the medical tests 115, instructions on their proper usage, and a clear explanation of the test results can be provided by the medical station. A customer could use the test improperly resulting in a non-responsive or unexpected result that triggers the system to provide the next level of support 115. The medical station provides further instructions on the usage of the test and re-dispenses the test, if needed. In addition, the test could give a result that has not been seen by the customer (by checking against the electronic patient record 230). In this case, the medical system performs a simple check with the customer as to their understanding of the results. Any further actions to be taken can be relayed to the appropriate medical professional, including a clerk, technician, nurse, pharmacist or doctor, either live or via the teleconferencing system.

In some cases, a higher level of medical assistance 120 will be triggered due to the results of the current medical test coupled with knowledge of the user's prior medical conditions or medications extracted from the patient record 230. For example, if a patient has a prior condition, such as diabetes, (as noted by the patient record), a blood glucose or stress test may indicate the need for immediate medical attention as opposed to a similar result from a previously healthy patient that may simply indicate the need to see a doctor sometime in the neat future.

At the highest level of support, specialist assistance is required 125. This can occur when a customer is under the care of a specialist who wishes to be aware of the test results immediately. In this embodiment, the specialist's e-mail address or other contact information is obtained (either from the patient or their medical record 230), and an urgent e-mail or text message to a portable messaging system such as a cell phone, PDA, pager or the like, is sent to the specialist. The specialist can respond via phone using the speaker/microphone on the medical station conferencing system, a text messaging system, or an Internet conference to the medical station. In another embodiment, contact is through simple phone communications from the central server 260 and the specialist. Alternatively, a customer support person located at the central server could facilitate communication between the user and their specialist.

FIG. 2 shows the system diagram. A medical testing system 200 typically has a computer, screen and pointing method (mouse or touch screen). In addition, the current invention has local storage 240 for temporary storage of the medical test results, a microphone 205, speaker 220, camera 210, and a means for reading an electronic patient record 230. This medical testing system 200 communicates to a central server 260 via the Internet, a local area network, a wide area network 250 or the like. The central server is located at the communications center 290 for the medical testing service. The central server 260 provides for Level 1 Support 130 and some Level 2 Support 135 as well as the means to communicate to additional Level 2 Support 135 and Level 3 Support 140 not necessarily, but in some cases, located at the communications center 290. The technical support 270 is usually on site, but can also be remote as shown by typical CPU support systems available today in most corporate environments. Some medical assistance 275 will be resident at the communications center 290. Note that the communications center 290 can have virtual connectivity and still be part of the infrastructure of the center. Medical professionals will handle some assistance remotely as they are needed 280. A database of medical professionals is provided to automatically find any available professional at the time of need. The doctor level service 280 and specialist level service 285 will most likely be handled in the latter manner as stated above, although there may be occasion to have some of these professionals employed at the communications center 290.

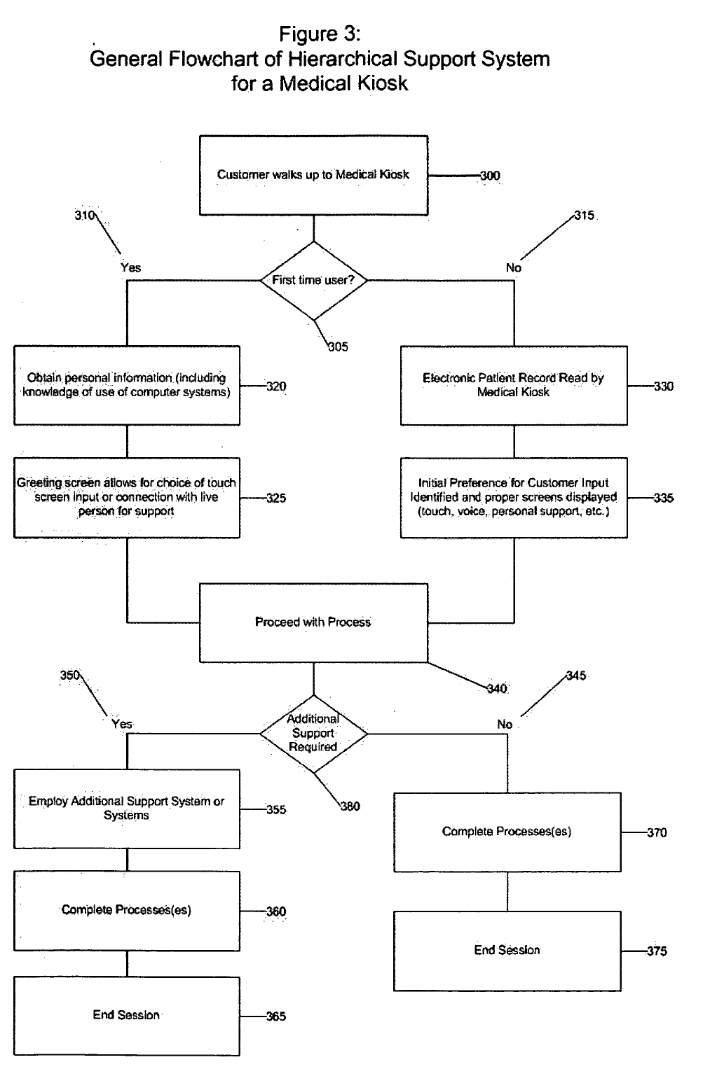

FIG. 3 shows the system workflow. A customer approaches 300 the medical testing system. An electronic sensor or touch area on the screen asks if this is the first time the customer 305 has used the station. If this is the first time 310, the customer is requested to provide information needed to use the medical system 320. This information includes their knowledge of computer systems and derives the level of support needed for the first time. After this information is collected, a greeting screen 325 will appear which allows the user to select the type of interaction, if they wish to override the choice made for them. If this is not the first time for this customer 315 or they have a compatible patient record that can be used in the station, they are instructed to have their electronic patient record (EPR) read by the system 330. Note that the EPR card read would override the screen query to determine if this is the first time the station is being used. The card contains the information on their preferences for the interaction 335 and a choice to allow them to change the interface for this session. After the method of interaction is decided, the system allows the requested test to be dispersed or implemented 340. Results from the test are automatically stored by the system into its local storage 240 or sent over the Internet 250 to the communications center 290 for processing, storage and forwarding. The medical system then checks to see if there is a need for additional assistance 380. If assistance is required at the time of testing or after the test is performed 350, the assistance system is automatically implemented and the proper level of assistance is employed 355. This is done until the process is completed 360 and the session ends 365. If no assistance is required 345, the process proceeds 370 until the session is completed 375.

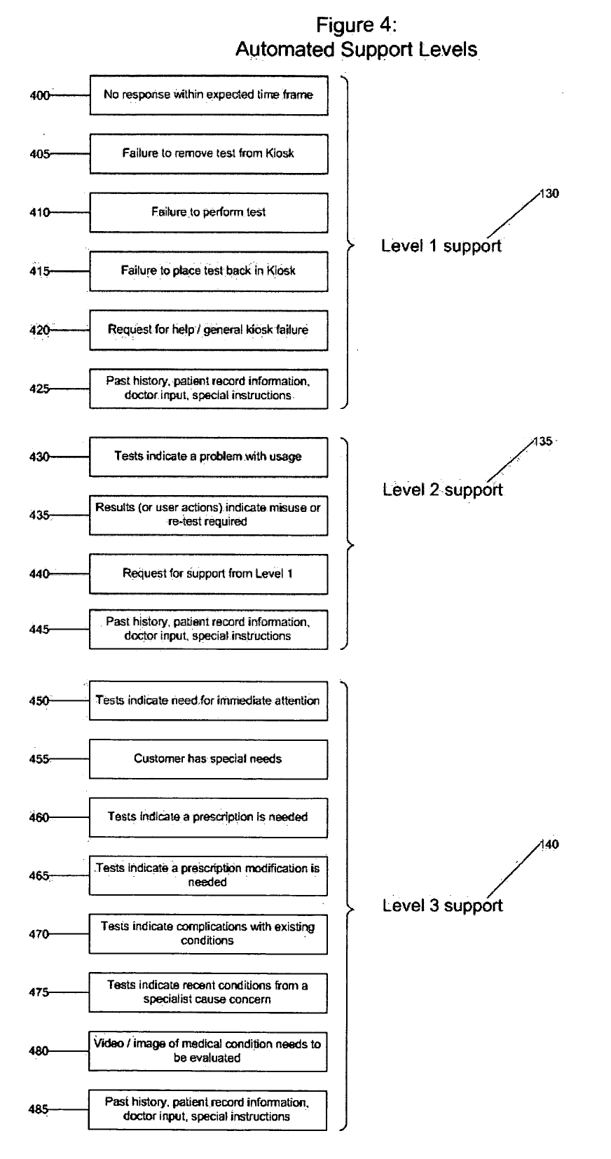

FIG. 4 illustrates the different types of problems that can occur within a given level of support. Level 1 Support 130 is provided under the following circumstances:
The customer does not respond to the prompts or requests within the expected time frame 400. In the preferred embodiment, a timer is set with each of the expected responses that are requested by the medical system. A window would appear with a person inquiring if assistance is needed. A reminder prompt may be used before the actual appearance of an assistant.
When a test is dispensed, the customer fails to retrieve the test from the medical system 405. In the preferred embodiment, a sensor is placed within the dispensing unit of the medical system. Failure to remove the test signals the medical system (and the communiations center) that the test is still in the dispenser for longer than expected. An indicator (light or touch screen icon) on the dispenser can be used to indicate when the test is available to the user.
If a test has been removed from the medical system and the test is not performed 410, assistance can be provided. This can automatically be accomplished via a RFID tag on the test indicating its use and wireless communications back to the medical systems.
If the test is not placed back into the system for analysis 415, technical assistance can automatically be signaled. Again, a sensor on the receptor part of the medical station allows for this information to be made available without user asking for assistance. A timer can also be utilized for this instance.
If the medical system has a general failure 420, it can generate a request for assistance. This is accomplished by a periodic status check signal sent from the central server to the medical station. If the proper signal is not returned, assistance to remedy the situation is initiated.
A medical record can contain information on past usage and interactions with medical testing systems as well as patient and medical professional preferences for such interactions. If this information indicates that the customer prefers to be assisted with the test from the beginning of their session, assistance can be automatically started as soon as the process starts 425. In addition, a doctor or other medical professional can state that assistance will be required for a particular user.

Some Level 2 Support 135 can automatically be provided. Level 2 Support requires technical assistance 110 of a higher degree that Level 1 and could involve addition training in the test aspect of the service. Instances where this is applicable include:
The results indicate a problem in the administration of the test or the results are questionable as to the expected results 430. The medical testing system or central server can analyze the test results for outliers and provide appropriate assistance, without a specific request from the customer.
The results are questionable as to the point where a re-test is required, or the test was damaged in some manner and assistance will alleviate this happening another time 435. The expected results (or lack of any results at all) are not seen by the medical testing system, which signals the communication center for assistance to the customer.
Level 1 Support may be unable to solve a problem due to lack of knowledge 440. It can be elevated to the next level of support 445 by any number of means currently used in computer support centers (call forward, automated computer databases or the like).
As with Level 1 Support, medical professionals can indicate via the patient medical record that Level 2 Support is required for a user. The communication center is notified of this requirement when the EPR is read, and Level 2 Support is initiated at the beginning of the test.

Level 3 Support 140 requires some level of medical assistance. This level of support can also be automatically provided to the customer through the medical system. Some of the instances where this would be desirable include:
Test results at the medical system indicate an emergency situation (for example, very high blood glucose levels to a diabetic or extremely high BP to a cardiac or hypertensive patient). In these cases, immediate attention by a medical professional is required 450. The medical professional can communicate the need to see a physician, change medication, re-do the test, or have the patient perform a specific task (lie down, seek emergency care, etc.)
The electronic patient record can indicate that the customer has special needs requiring a medical support person to be aware the testing is being done 455. Examples are asthmatics performing a breath test that could cause an attack or someone needing special support in using the tests (trained personnel who handle disabilities).
The results of the test indicate that a prescription 460 is needed or needs to be modified 465. In this case, a doctor or pharmacist is required. Communications with the appropriate medical professional can be automatically established (as well as an e-prescription sent to the pharmacy of choice).
Certain medical conditions can be of concern when a test is being performed. The aforementioned asthmatic or coronary patients are examples. Even a test result only slightly elevated may be recorded, but it may cause a trigger for concern about another condition 470. Since the condition can be found on the EMR, a special set of limits on the test can be initiated. If concern is high enough (as stated by the customer's caregiver), an automated signal for medical assistance can be enabled.
A recent condition (surgery, psychological trauma, or the like) may cause the patient's specialist to want to be aware of the test results 475. Again, the EMR would contain this information. The appropriate specialists can be contacted and communications established if a concern arises over the test results (or even the test itself being conducted). The specialist can send a message to the station or even appear on screen via the teleconferencing capability of the station.
A customer at the station could be in distress as indicated by the results of the medical tests. A video link to the control center can provide a 2-way video conference 480 between the customer and a medical professional, even the customer's own doctor.
The customer's past history indicates that medical assistance should be available while the test is being performed 485. This could also be at the request of the customer and at an additional charge. This can be on the EMR, at the touch of an option on the screen, or a verbal request to one of the other support levels.

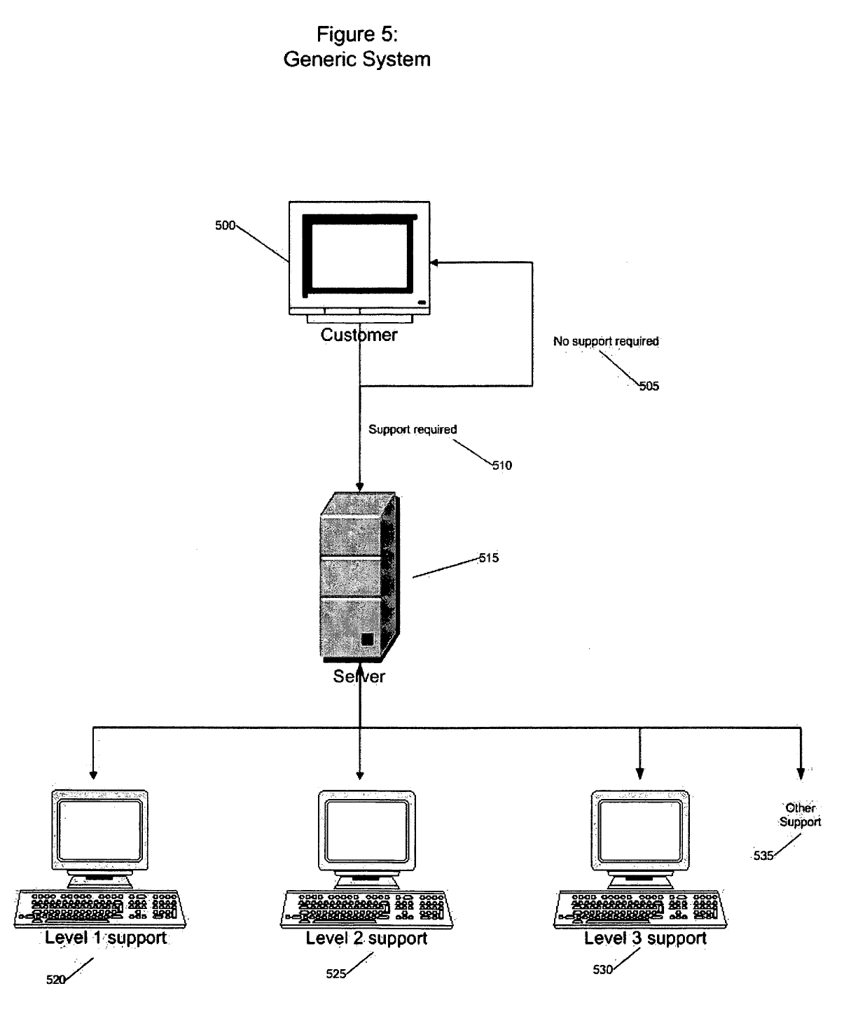

FIG. 5 shows a generic system set-up for such a support system. The customer terminal 500 can be at a medical station or at any Internet-enabled computer. If no assistance is required 505, the customer simply proceeds. If assistance is required 510, a server 515 channels the request to the proper levels of support 520, 525, and/or 530. It is important to note that Level 2 Support 525 does not need to go through Level 1 Support 520 to be utilized. The same is true for Level 3 Support not requiring Level 1 Support 520 or Level 2 Support 525 to be implemented first. Other levels of support 535 (perhaps a link to a family member or care facility) are also possible via this architecture.

The invention has been described in detail with particular reference to certain preferred embodiments thereof, but it will be understood that variations and modifications can be effected within the scope of the invention.

### PARTS LIST

100 standard touch screen support
105 live clerical service
110 live technical service
115 basic medical service
120 doctor level service
125 specialist level service
130 Level 1 Support
135 Level 2 Support
140 Level 3 Support
200 medical kiosk or medical station
205 microphone
210 camera
220 speaker
230 electronic patient record
240 local storage
250 communications mode
260 central server and control center
270 tech assistance center
275 medical assistance center
280 doctor assistance center
285 specialist assistance center
290 communications center
300 customer at medical station
305 is a first time user?
310 decision as to first time user
315 is not a first time user?
320 obtain preference information
325 allow for choice of interface
330 read EPR
335 initial choice for user preference and option to change
340 perform test
345 no additional assistance required
350 additional assistance required
355 employ assistance system
360 complete tests with assistance
365 end session with assistance
370 complete tests without assistance
375 end session without assistance
380 is additional assistance required?
400 time out
405 test not removed
410 test not performed
415 failure to return test
420 help with station failure
425 past history requires clerical assistance
430 problem with using the test
435 test is misused/re-test required
440 request from clerical level
445 past history requires technical assistance
450 immediate medical attention required
455 special needs
460 prescription needed
465 prescription modification needed
470 complications with existing conditions
475 recent specialist level conditions
480 video evaluation
485 past history requires medical assistance
500 customer terminal
505 no assistance required
510 assistance required
515 central server
520 Level 1 Support
525 Level 2 Support
530 Level 3 Support
535 other levels of support

1. A method of providing automated assistance at a remote medical testing system comprising:
using hierarchical levels of support based on intelligent use of historical patient data and contextual awareness of events as they are occurring.

2. A method as in claim 1 comprising the additional steps of:
using predefined logic to escalate users to the appropriate level of automated support.

3. A method as in claim 1 comprising the additional steps of:
using predefined logic to escalate users to the appropriate level of live technical support.

4. A method as in claim 1 wherein said logic and contextual awareness is used to connect the user to the appropriate level of live medical assistance.

5. A method of providing automated assistance at medical station comprising:
(a) initiating interaction between said medical station and a patient;
(b) entering patient identification information;
(c) determining if said patient is a first time user;
(d) if said patient is a first time user go to step (e) if not go to step (f);
(e) entering additional patient information;
(f) accessing a patient medical record;
(g) testing said patient;
(h) determining if a higher level of support is needed;
(i) if a higher level of support is needed go to step (j), if not end procedure; and
(j) providing a higher level of support.

6. A method as in claim 5 wherein said initiation of interaction between said medical station and said patient is by means of an electronic patient record card.

7. A method as in claim 5 wherein said interaction step is accomplished by entering data with a keyboard, data entry, or touch screen.

8. A method as in claim 5 wherein said testing step is comprised of administering at least one medical test.

9. A method as in claim 5 wherein said testing step is comprised of administering at least one medical test and visual observation of said patient.

10. A method as in claim 5 wherein said step of determining if said higher level of support is needed is comprised of a computer algorithm for weighting results of at least one medical test, visual observation of said patient, and said patient medical record.

11. A method as in claim 5 wherein said step of providing said higher level of support includes dispatching emergency medical personnel to said station.
